(19) United States
(12) Patent Application Publication
(10) Pub. No.: US 2009/0182190 A1
(43) Pub. Date: Jul. 16, 2009

(54) Surgical Tools and Methods for Treating Incontinence and Related Pelvic Conditions

(75) Inventor: Jeffrey A. Dann, Ormond Beach, FL (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/352,164

(22) Filed: Jan. 12, 2009

Related U.S. Application Data
(60) Provisional application No. 61/020,535, filed on Jan. 11, 2008.

(51) Int. Cl. A61F 2/02

(52) U.S. Cl. 600/30; 600/37

(57) ABSTRACT

Pelvic implant system having a sling implant that can be inserted into a patient's pelvic cavity with an introducer needle device. The introducer needle device includes a handle portion having an actuator. An elongate needle portion extends away from the handle and has an engagement hook proximate a distal end of the elongate needle portion. The engagement hook is capable of selectively engaging a portion of the sling implant upon activation of the handle actuator.

### PRIORITY APPLICATIONS

This application claims the benefit of and priority to U.S. Provisional Application No. 61/020,535, filed Jan. 11, 2008, the disclosure of which is incorporated by reference herein in its entirety.

### FIELD OF THE INVENTION

The invention relates to apparatus, tools and methods for treating pelvic conditions by use of a pelvic implant to support pelvic tissue. The pelvic conditions include conditions of the female or male anatomy, and specifically include treatments of female or male urinary and fecal incontinence, and treatment of female vaginal prolapse conditions including enterocele, rectocele, cystocele, vault prolapse, and any of these conditions in combination. In particular, the present invention relates to tools for facilitating implantation of surgical implants that support pelvic tissue and that are secured in the abdominal area.

### BACKGROUND

Pelvic health for men and women is a medical area of increasing importance, at least in part due to an aging population. Examples of common pelvic ailments include incontinence (fecal and urinary) and pelvic tissue prolapse (e.g., female vaginal prolapse). Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

In its most severe forms, vaginal vault prolapse can result in the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons. These procedures often involve lengthy surgical procedure times.

Urinary incontinence can be characterized by the loss or diminution in the ability to maintain the urethral sphincter closed as the bladder fills with urine. Male or female stress urinary incontinence (SUI) occurs when the patient is physically stressed.

One cause of urinary incontinence is damage to the urethral sphincter. Other causes include the loss of support of the urethral sphincter, such as can occur in males after prostatectomy or following radiation treatment, or that can occur due to pelvic accidents and aging related deterioration of muscle and connective tissue supporting the urethra. Other causes of male incontinence include bladder instability, over-flowing incontinence, and fistulas.

The female's natural support system for the urethra is a hammock-like supportive layer composed of endopelvic fascia, the anterior vaginal wall, and the arcus tendineus. Weakening and elongation of the pubourethral ligaments and the arcus tendineus fascia pelvis, and weakening of the endopelvic fascia and pubourethral prolapse of the anterior vaginal wall, may have a role in the loss of pelvic support for the urethra and a low non-anatomic position that leads to urinary incontinence.

In general, urinary continence is considered to be a function of urethral support and coaptation. For coaptation to successfully prevent or cure incontinence, the urethra must be supported and stabilized in its normal anatomic position. A number of surgical procedures and implantable medical devices have been developed over the years to provide urethral support and restore coaptation. Examples of such surgical instruments included Stamey needles, Raz needles, and Pereyra needles. See Stamey, Endoscopic Suspension of the Vesical Neck for Urinary Incontinence in Females, Ann. Surgery, pp. 465-471, October 1980; and Pereyra, A Simplified Surgical Procedure for the Correction of Stress Incontinence in Women, West. J. Surg., Obstetrics & Gynecology, pp. 243-246, July-August 1959. Other treatments involve implantation of a Kaufman Prosthesis, an artificial sphincter (such as the AMS-800 Urinary Control System available from American Medical Systems, Inc.), or a urethral sling procedure in which a urethral sling is inserted beneath the urethra and advanced to the retropubic space. Peripheral or extension portions of the elongated urethral sling are affixed to bone or body tissue at or near the retropubic space. A central support portion of the elongated urethral sling extends under the urethral or bladder neck to provide a platform that compresses the urethral sphincter, limits urethral distention and pelvic drop, and thereby improves coaptation. Similar attached slings or supports have been proposed for restoring proper positioning of pelvic organs, e.g., the vagina or bladder. In other procedures, autologous fascia was used as an artificial graft material that was placed under the bladder neck and tied or anchored to the pelvic bone.

### SUMMARY

The present patent application describes pelvic implants and methods for treating pelvic conditions such as incontinence (various forms such as fecal incontinence, stress urinary incontinence, urge incontinence, mixed incontinence, etc.), vaginal prolapse (including various forms such as enterocele, cystocele, rectocele, vault prolapse, etc.), among others. Embodiments of the various tools or introducers as well as the surgical techniques are described herein.

According to the invention, a sling, implant tool and method are being proposed that eliminate some of the challenges of the prior art. This involves placement of the graft material under the mid-urethra and not the bladder neck and so as to bring the “entire” graft material up to the abdominal wall fascia via a blind percutaneous approach thereby simplifying and securing the support of the bladder neck with a long hammock that transverses the endopelvic fascia and abdominal wall fascia. Although preferable in theory, instruments have been scarce that specifically facilitate retrieval and correct placement of the graft material and noninvasive anchoring of the graft to the abdominal wall. We propose such a surgical kit that would facilitate improved placement of the graft or sling material in the mid-urethra, via an easy percutaneous retrieval of the graft to the abdominal wall and adequate attachment of the graft material to the anterior rectus fascia avoiding suture slippage and long term infections. Therefore, we prefer that this novel introducer tool be packaged such that it is available in a sterilized form in a disposable kit.

Incontinence device kit of the invention includes all the materials necessary to perform a transvaginal sling procedure: 1) a disposable passer having an ovoid tip which permits penetration of the abdominal fascia retroperitoneal tissue and endopelvic fascia with adequate dilation of the endopelvic fascia to accept sling material. The tip of the passer is adapted to easily accept the end of the graft material so that it can atraumatically brought to the abdominal wall; 2) nonabsorbable graft material with a hole at the distal end so as to fit into the passer; 3) a calibrated catheter to permit localization of the correct incision over the urethra; 4) a water soluble button or clip or funnel through which the graft is placed. The graft is secured into the button and the button is then secured to the anterior abdominal wall fascia causing the graft to be securely fixed and prevents migration into the periostinium. The material is slowly absorbed over a fixed period or amount of time so that tissue ingrowth can secure the sling in place over time.

The invention also contemplates a method of treating urinary incontinence in male and female patients. The method includes creating at least one medial incision (a transvaginal incision or a perineal incision) under the mid-urethra, dissecting a tissue path on each side of the incision, passing a urinary incontinence sling through the incision whereby the urinary incontinence sling is suspended in the retropubic space or endopelvic fascia such that the sling body is positioned between the patient's urethra and vaginal wall (for a female) to provide support to the urethra and optionally exits at least one suprapubic or prepubic incision. For males, a perineal incision can be made to pass the sling through the incision and suspend the sling in a manner comparable to the sling installed in the female patient anatomy. A procedure for treating male urinary incontinence may be performed with or following a prostatectomy, or otherwise.

In addition to treating urinary incontinence, the invention also contemplates methods relating to other types of pelvic floor repairs. Currently, pelvic floor repairs are surgically treated through graft augmented repairs and with kit systems that use needles to deliver a graft through an incision on the anterior and posterior vaginal wall. These current procedures address tissue, muscle and ligament weakness in the pelvic floor such as rectoceles, enteroceles, cystoceles, apical, fecal and flatal incontinence, perineal and uterine descent.

Another aspect of the invention includes a combination (e.g., kit, system, etc.) of an implant, as described herein and which can be fashioned by the physician during surgery, along with a surgical tool or introducer for implanting the implant.

In another aspect, the invention relates to a method of treating a pelvic condition. The method includes providing an implant according to the current description; providing an insertion tool that includes a handle and a needle extending from the handle, the needle including a proximal end attached to the handle and a distal end, the distal end including a needle having at least one hook for engaging an implant. In a related embodiment, the implant tool includes a balloon dilator along the length of the needle that is inflatable to dilate tissue in the pelvic/abdominal area when passing the needle through or when locating a pelvic implant. The balloon dilator can also be used in the implantation of other incontinence devices such as an artificial urinary sphincter or for facilitating implantation of bulk implants or bulking agents.

### BRIEF DESCRIPTION OF DRAWINGS

Other features and advantages of the present invention will be seen as the following description of particular embodiment's progress in conjunction with the drawings. Drawings are schematic and not to scale.

FIG. 1 illustrates one embodiment of the surgical tool kit for implanting a pelvic implant.

FIGS. 2A-2C are schematics of the top, side and expanded views of the needle portion and needle end of the surgical tool/introducer of FIG. 1.

FIGS. 3A & 3B illustrate one embodiment of a balloon dilating introducer, tip operation and mesh capturing device according to the invention.

FIG. 4 illustrates an embodiment of a pelvic implant kit that includes an implant tool according to the invention.

FIGS. 5A-5G illustrate the various steps of the surgical method for locating an implant using the implant tool of FIG. 3.

### DETAILED DESCRIPTION

The following description is meant to be illustrative only and not limiting. Other embodiments of this invention will be apparent to those of ordinary skill in the art in view of this description.

The present invention is directed to surgical instruments, assemblies, and implantable articles for treating pelvic floor disorders such as fecal or urinary incontinence, including stress urinary incontinence (SUI), prolapse, etc. According to various embodiments, a surgical implant can be used to treat a pelvic condition, including the specific examples of implanting a support member (“implant”) to treat a condition such as vaginal vault prolapse or incontinence (male or female). Described are various features of surgical implants, surgical tools, surgical systems, surgical kits, and surgical methods, useful for installing implants. An implant can be implanted in a male or a female to treat disorders such as urge incontinence, mixed incontinence, overflow incontinence, functional incontinence, fecal incontinence, or for female conditions including prolapse (e.g. vaginal or uterine), enteroceles (e.g. of the uterus), rectoceles, cystocele, and anatomic hypermobility.

Exemplary implants can include a tissue support portion for placing in contact with tissue to be supported and one or more “extension” portions, the tissue support portion being useful to support a specific type of pelvic tissue such as the urethra, bladder, or vaginal tissue (anterior, posterior, apical, etc.). The tissue support portion can be sized and shaped to contact the desired tissue when installed, e.g., as a “sling” or “hammock,” to contact and support pelvic tissue. A tissue support portion that is located between two or more extension or extension portions is sometimes referred to herein as a “central support portion” or a “support portion.”

Extension portions are elongate pieces of material that extend from the tissue support portion and either are or can be connected to the tissue support portion, and are useful to attach to anatomical features in the pelvic region to thereby provide support for the tissue support portion and the supported tissue. One or multiple (e.g., one, two, or four) extension portions can extend from the tissue support portion as elongate “ends,” “arms,” or “extensions,” useful to attach to tissue in the pelvic region, such as by extending through a tissue path to an internal anchoring point as described herein.

Types of exemplary implants that can be generally useful as discussed herein can include those previously and currently used in treating pelvic conditions, including those implants referred to as urethral “slings,” “strips,” “mesh strips,” “hammocks,” among other terms for pelvic implants.

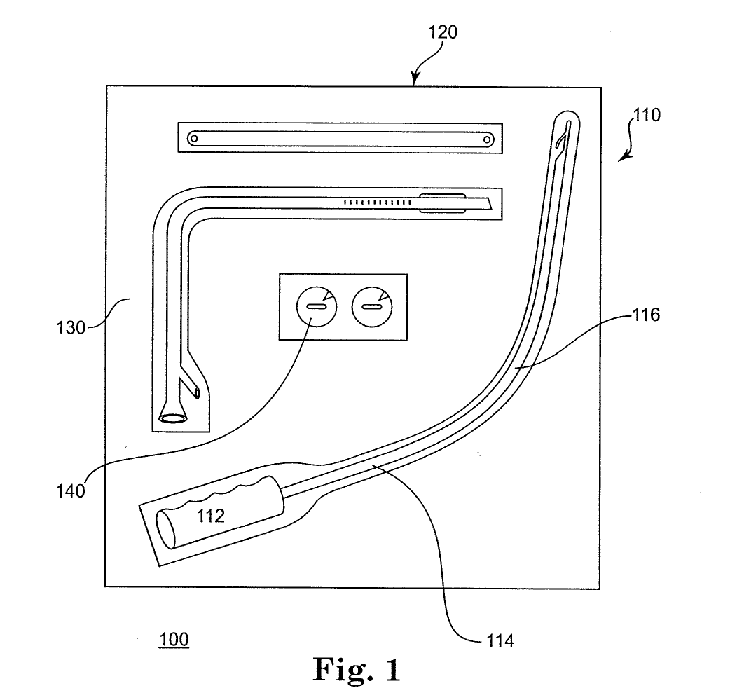

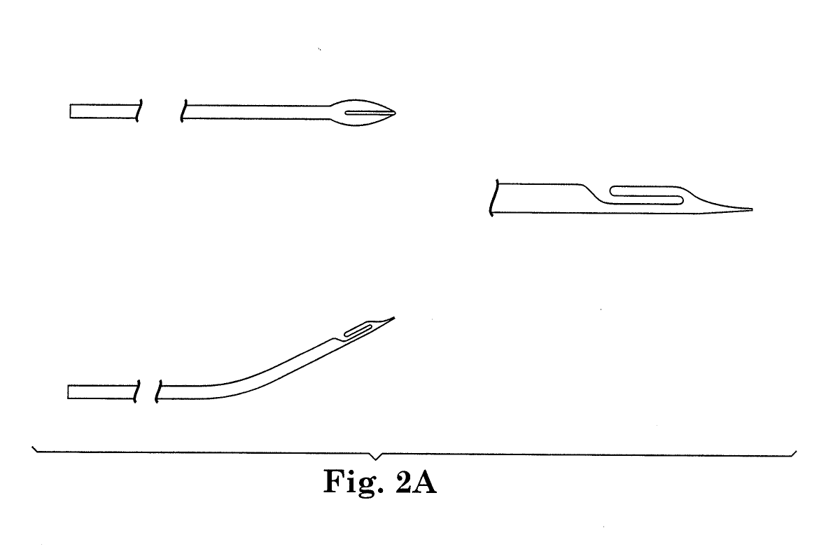

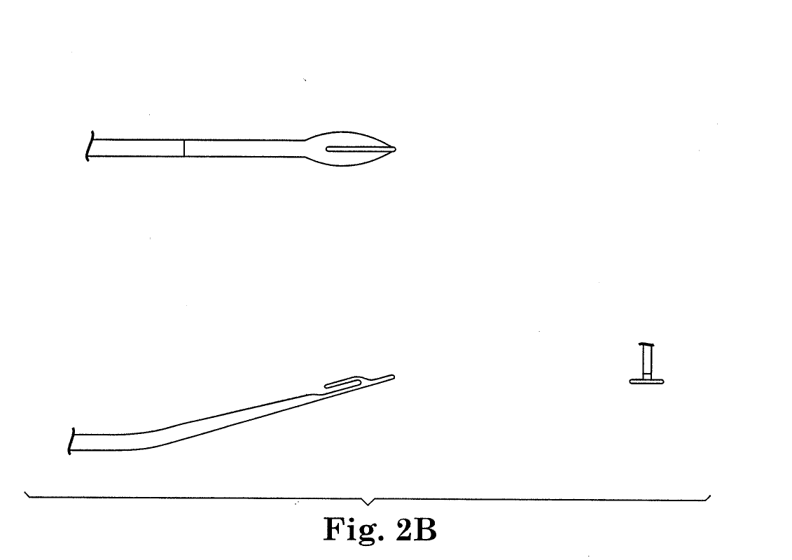

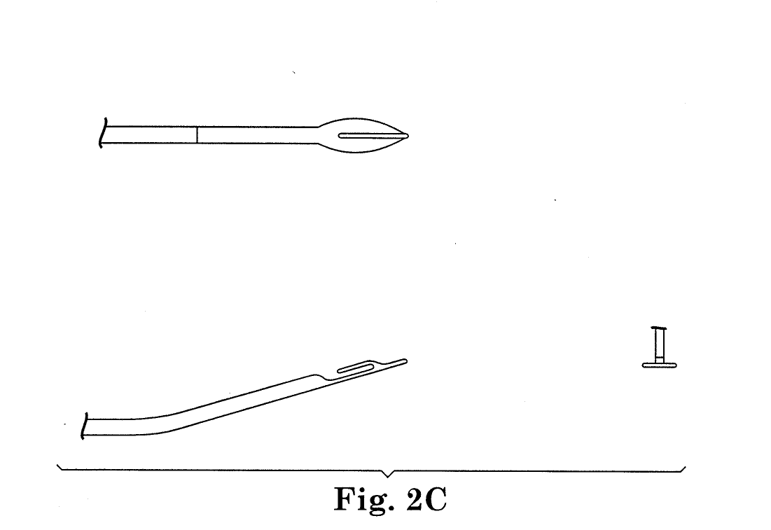

Referring now to the Figures, FIG. 1 and 2A-2C illustrate one embodiment of the surgical tool kit 100 for implanting a pelvic implant (transvaginally or transperineally) as well as a schematic of the top, side and expanded views of the needle portion and needle end of the surgical tool/introducer of FIG. 1. In this embodiment, kit 100 includes four (4) instruments: a passer (110), sling or mesh material (120), a water soluble fascial anchor (not shown) such as a button or clip, and a calibrated catheter (130) to measure urethral length, and optional instructional tape or video (140). The passer consists of a handle (112), curved metal shaft (114), and unique bend (116). The handle is shaped to be ergonomically favorable and to permit easy leverage of the device as it is passed percutaneously through the abdominal wall tissue. The handle indentations (113) permit an easy grasp of the handle. The metal shaft is shaped so as to pass along the pubic bone and not perforate the underlying bladder. The angle of the distal portion of the shaft can vary from 0 degrees to 90 degrees.

The bend of the passer is uniquely shaped in an ovoid geometry to protect adjacent organs and to avoid the needle from straying away from the preferred path. This permits easy passage through the endopelvic fascia with minimal damage to the surrounding tissue or bleeding. The shape and size also permits dilation of the endopelvic fascia to adequately pull the graft material through the endopelvic fascia. Although it is preferred as an ovoid geometry, any atraumatic geometry can suffice, including bulbous or circular. In its preferred embodiment, the head and hook are parallel with the plane of the shaft; the hook being co-linear with the shaft. At the tip of the head is a sharp protuberance. This sharp protuberance provides a point for piercing the abdominal wall fascia, underlying pelvic tissue and endopelvic fascia.

The graft material or sling 120 must be of sufficient length to reach the abdominal wall on both sides. The material can consist of various autologous and biologic materials including autologous fascia, bovine fascia, absorbable mesh, a non-absorbable mesh such or polypropylene. The mesh width can also vary from 2-3 centimeters. At each end of the mesh is a hole which permits the passer 110 to engage the mesh and pull it upwards to the abdominal wall. In its preferred embodiment this is a hole although it can also be a closed loop (or other shapes that permit engagement with the hook at the needle end) that protrudes from the end of the tape.

The urethral catheter is also included. The catheter is placed into the bladder to measure urethral length. The surgeon may then know how far away from the top of the urethra in order to make the incision so that the sling can be correctly placed in the mid-urethral section. The abdominal button serves as an anchor to prevent the sling from falling back into the retropelvic or retropubic space. Although this clip device can be constructed of non-absorbable material, absorbable material is its preferred embodiment. It can be constructed from a water soluble polypropylene which gradually dissolves over a predetermined period of time. The button has various shapes and sizes but in its preferred embodiment is a truncated graduated shape with the narrow end facing down towards the pelvic floor (like an upside down funnel or cone). In the center of the button is a slit, slot or hole which is of sufficient size to permit passage of the graft material through the center of the button.

In the preferred embodiment, the button is constructed of a water soluble material which dissolves over a period of 1-2 months. Water soluble material provides a stable fixation for a period of several weeks, allowing the graft material to become incorporated into the surrounding tissue. After this period is allowed, the water soluble material should become absorbed so that within several months it has disappeared, thereby eliminating the need for post-operative removal of the button and avoiding infection from a permanent fixation device. The material utilized to construct the clip or button includes polypropylene.

The surgical kit of the invention would permit easy access by the surgeon to each of these materials needed to conduct the surgery. The kit would come in a sterile, disposable tray or holder, including the tissue passer, fashioned or customizable graft material, calibrated urethra catheter and water soluble fascial buttons for fixation.

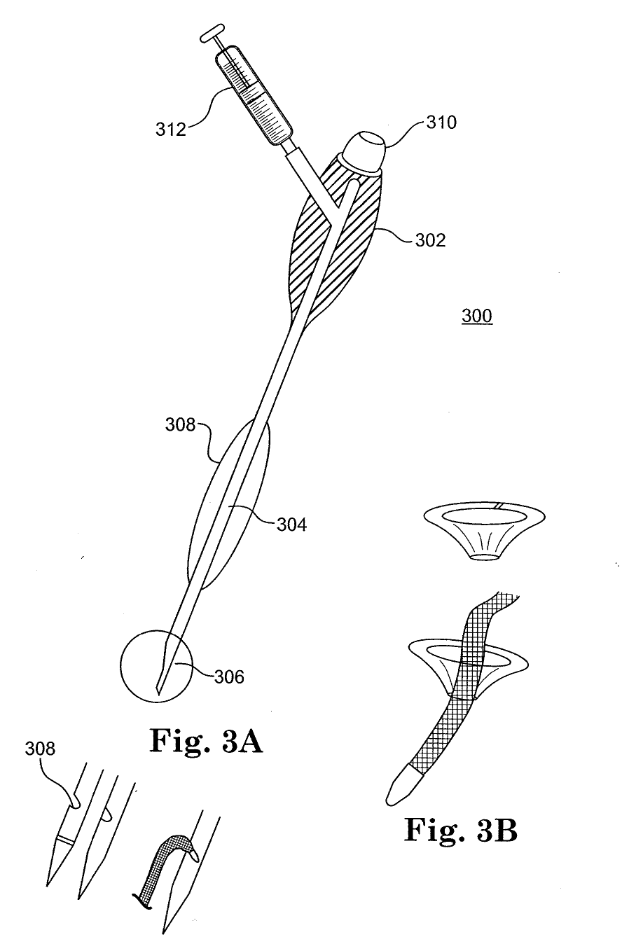

In another embodiment of the invention, FIGS. 3A and 3B illustrate one embodiment of a balloon dilating introducer 300. Introducer 300 includes a tip that operates in a manner that facilitates various pelvic implant treatments including, but not limited to, urinary and fecal incontinence and other pelvic conditions in both men and women.

Referring further to FIGS. 3A and 3B, dilator tool or introducer 300 includes a handle 302, an elongated portion 304, and a needle tip 306 with an engageable hook portion 308 that is activated by a button 310 located on handle 302. Handle 302 further includes syringe type mechanism 312 to inflate a balloon dilation member 308 is located on or about the elongate portion of the needle. Dilator member 308 is located proximal to tip 306. FIG. 3A illustrates how needle tip 306 can be opened and closed to engage the mesh or closed to avoid engaging tissue while the needle is being passed through the pelvic tissue. Dilator tool 300 facilitates the implantation of meshes or other devices by dilating tissue as the tool is introduced or when the tool is being pulled from the patient, so dilation can occur in both directions depending upon the physician's needs and choice. FIG. 3B illustrates how a mesh or sling can be captured by a button or clip device at or near or below the skin and in the fascia, to prevent sling slippage after surgery.

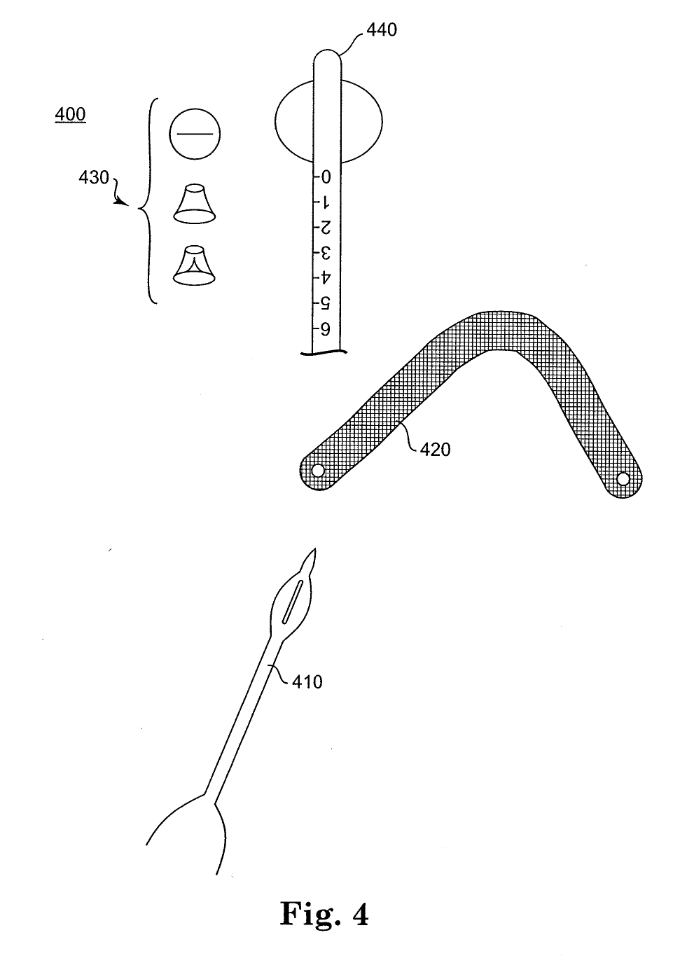

FIG. 4 illustrates an embodiment of a pelvic implant kit 400 with the implant tool 410, a sling or mesh implant 420, abdominal clips, buttons and/or funnels 430, a urethral catheter 440 for calibrating the urethral length and a balloon dilator tool of according to the invention.

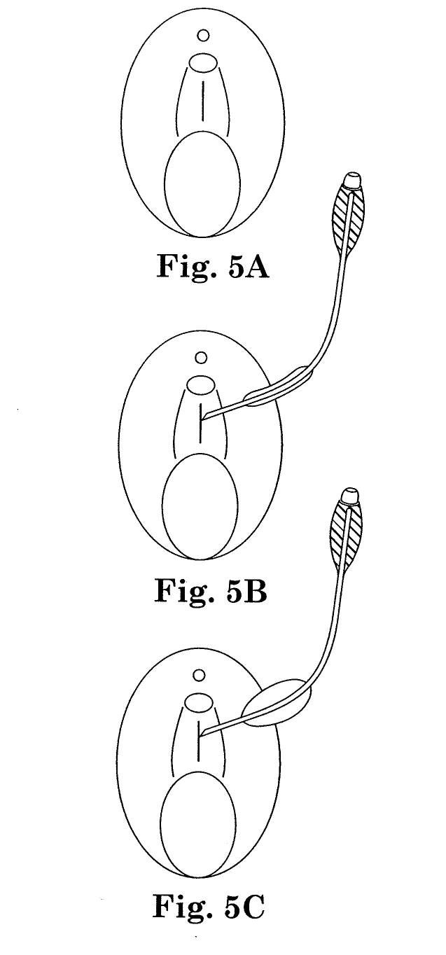

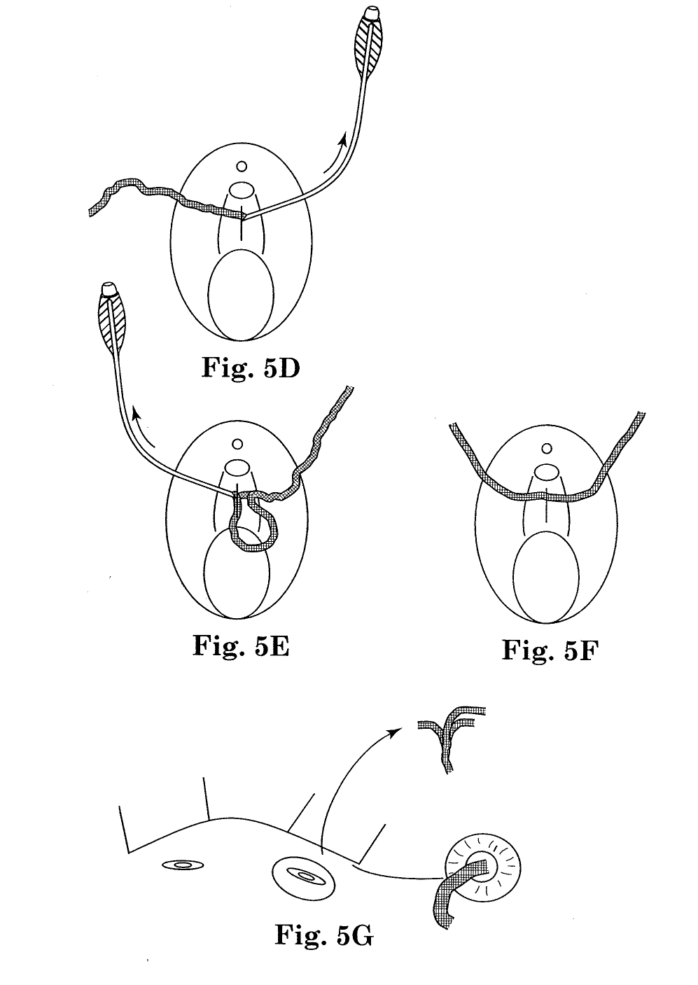

Referring now to FIGS. 5A-5G, there is illustrated the various steps of the surgical method for locating an implant using the implant tool 300 (or other tools described in this application). In the initial step, a urethral or medial incision is made under the urethra (clitoris is shown for positioning). Dilator tool 300 is then inserted uninflated or inflated through the incision so as to either pass the needle through or pass the needle and dilate tissue as the needle is being passed through. Suprapubic or prepubic incision or incisions can be made earlier in the procedure to allow for passage of tool 300 or the tool can puncture the abdominal skin from the inside when the needle is passed underneath. Once tool 300 exits the suprapubic incision, a sling or mesh can be pulled back through and out of the urethral or perineal incision. The step is repeated on the other side to form a U or V shape under the urethra or other tissue or organ that is being supported (such as the rectum). The mesh can be made from Martex or Prolene™ (polypropylene), a biologic material or autologous or cadaveric tissue. FIG. 5G illustrates a side view of the exit points of the mesh and how they can be held in place with clips or funnels or the like.

For a typical procedure for treating any pelvic condition, a patient may be first placed under local, spinal, or general anesthesia. According to exemplary methods of treating a female condition of incontinence (e.g., a small, medial, transvaginal incision for treating female urinary incontinence) is made in the upper wall of the vagina under the mid-urethra. For implantation of a sling to treat incontinence in a male, a perineal incision may be made instead. The incision should be large enough for the surgeon to place the sling through the incision using selected instruments. A desired amount of tissue may optionally be dissected on each side, for placement of sling. In one embodiment the tissue may be dissected approximately 1-2 centimeters in each direction away from the urethra.

As previously discussed, the sling or a portion of an implant may be positioned inside a sleeve before the implant is inserted through the incision. In alternate embodiments, sleeve may not be used or necessary, depending on surgeon preference. In one embodiment, sleeve or a delivery tool can cover the woven portion during implantation. As described herein, embodiments of the invention can involve the use of various types of delivery tools to prevent an extension portion of an implant from contacting tissue of a tissue path during insertion of the extension portion through a tissue path.

The precise anatomical position of an implant can depend on a variety of factors including the type and degree of anatomical damage, location of significant scar tissue, and whether the procedure is combined with other procedures. Typically, an implant such as a urethral sling (e.g., sling) can be placed mid-urethra, without tension, but in position to support the mid-urethra. Alternately, the sling could be placed to support the bladder neck and/or UV junction. Implants for use to treat prolapse can be positioned at the middle or posterior vagina, or vaginal vault. Implants for treating fecal incontinence can be placed in the posterior portion of the pelvic region to support tissue for treating fecal incontinence.

Sling tension may be adjusted by a tension member such as a tensioning suture disclosed, for example, in U.S. Pat. No. 6,652,450. The tensioning suture may be constructed from a permanent or absorbable (i.e., bioresorbable or bioabsorbable) material. In still further embodiments, an implant such as sling can be introduced with a desired amount of tension in a number of different ways, such as those discussed elsewhere in the present description. A plastic sleeve or sheath, if present, may be removed after implantation of an implant such as sling and before the adjustment of tension by a tension member such as a tensioning suture. Once the implant is positioned and optionally tensioned or adjusted, the incision may be closed.

Although embodiments of the present invention have been described with reference to the treatment of female urinary continence, it should be appreciated that many of these embodiments would also be suitable to repair a variety of pelvic conditions in both males and females. For example, embodiments of the present invention would be suitable for a variety of pelvic floor repairs and/or treatments, including pelvic organ prolapse repair, levator hiatus repair, fecal incontinence treatment, perineal body support and hysterectomy support.

The following patents and publications are also herein incorporated by reference in their entireties: US Publications 2002/0128670; 2003/0191480; 2005/0148813; and U.S. Pat. Nos. 6,506,190; and 7,131,944; and WO 2006/069078 A2.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A pelvic implant system, comprising:
a sling implant;
an introducer needle device including;
a handle portion having an actuator; and
an elongate needle portion having an engagement hook proximate a distal end of the elongate needle portion to selectively engage a portion of the sling implant upon activation of the handle actuator.

2. The pelvic implant system of claim 1, wherein the sling implant is constructed at least in part of a mesh material.

3. The pelvic implant system of claim 1, wherein the sling implant includes an aperture adapted to receive the engagement hook of the elongate needle portion.

4. The pelvic implant system of claim 1, wherein the introducer further includes a balloon portion disposed along a portion of the elongate needle portion.

5. The pelvic implant system of claim 4, wherein the handle includes a syringe mechanism in operable communication with and adapted to selectively dilate the balloon portion.

6. The pelvic implant system of claim 1, wherein the elongate needle portion is generally arcuate.

7. A pelvic implant system, comprising:
a sling implant;
an introducer needle device including;
a handle portion;
an elongate needle portion having a distal portion adapted to engage a portion of the sling implant; and
a ballooning mechanism disposed along a potion of the elongate needle portion and in operable communication with the handle to facilitate selective dilation of the ballooning mechanism.

8. The pelvic implant system of claim 7, wherein the handle further includes an actuator and the distal portion of the elongate needle portion includes a hooked engagement portion in operable communication with the actuator such that activation of the actuator selectively engages the sling implant.

9. The pelvic implant system of claim 7, wherein the sling implant is constructed at least in part of a mesh material.

10. The pelvic implant system of claim 7, wherein the handle includes a syringe mechanism in operable communication with and adapted to selectively dilate the ballooning mechanism.

11. The pelvic implant system of claim 7, wherein the elongate needle portion is generally arcuate.

12. A pelvic implant system, comprising:
an introducer needle device having;
a handle portion;
an elongate needle portion having a selectively engageable hook proximate a distal end of the elongate needle portion; and
a ballooning mechanism disposed along a potion of the elongate needle portion and in operable communication with the handle to facilitate selective dilation of the ballooning mechanism.

13. The pelvic implant system of claim 12, further including a sling implant adapted for engagement with the selectively engageable hook of the elongate needle portion.

14. The pelvic implant system of claim 12, wherein the handle portion includes an actuator in operable communication with the selectively engageable hook.

15. The pelvic implant system of claim 12, wherein the handle portion includes a syringe mechanism in operable communication with the ballooning mechanism to facilitate selective dilation of the ballooning mechanism.

16. The pelvic implant system of claim 12, wherein the elongate needle portion is generally arcuate.

17. The pelvic implant system of claim 12, further including a sling implant constructed at least in part of a mesh material.

18. The pelvic implant system of claim 17, wherein the sling implant includes an aperture adapted to mate with the selectively engageable hook of the elongate needle portion.

19. The pelvic implant system of claim 17, wherein the sling implant is adapted to provide pelvic tissue support.

20. The pelvic implant system of claim 17, wherein the sling implant includes one or more soft tissue anchors.
